(19) United States
(12) Patent Application Publication
(10) Pub. No.: US 2013/0129780 A1
(43) Pub. Date: May 23, 2013

(54) MODIFIED INFECTIOUS LARYNGOTRACHEITIS VIRUS (ILTV) AND USES THEREOF

(71) Applicants: Maricarmen Garcia, Athens, GA (US); Alice Mundt, Watkinsville, GA (US); Egbert Mundt, Watkinsville, GA (US)

(72) Inventors: Maricarmen Garcia, Athens, GA (US); Alice Mundt, Watkinsville, GA (US); Egbert Mundt, Watkinsville, GA (US)

(73) Assignee: UNIVERSITY OF GEORGIA RESEARCH FOUNDATION, INC., Athens, GA (US)

(21) Appl. No.: 13/813,838

(22) PCT Filed: Aug. 2, 2011
(86) PCT No.: PCT/US11/46263

§ 371 Date: Feb. 1, 2013

Related U.S. Application Data
(60) Provisional application No. 61/369,986, filed on Aug. 2, 2010.

(51) Int. Cl. A61K 39/12; A61K 35/76

(52) U.S. Cl. CPC A61K 39/12; A61K 35/76
USPC 424/205.1; 435/236; 424/93.2

(57) ABSTRACT

Provided herein are modified infectious largotracheitis viruses (ILTVs) and methods of using the same. For example, provided are attenuated ILTVs. The attenuated ILTVs can be used to illicit immune responses in avian species. Optionally, the attenuated ILTVs can be used to vaccinate an avian subject or a population of avian subjects. Optionally, an attenuated ILTV is administered in ovo to an avian egg. One or more such in ovo administration can be used to increase the immunity of an avian herd.

### CROSS-REFERENCE TO RELATED APPLICATIONS

This application claims the benefit of U.S. Provisional Application No. 61/369,986, filed on Aug. 2, 2010, which is incorporated by reference herein in its entirety.

### BACKGROUND

Infectious laryngotracheitis (ILT) is a highly contagious respiratory disease of chickens that causes severe production losses to the poultry industry. The etiological agent for ILT is Infectious laryngotracheitis virus (ILTV).

The main sites of ILTV replication are the larynx, trachea and conjunctiva. Severe clinical signs are observed as respiratory manifestations such as gasping, coughing, expectoration of bloody mucus, and suffocation. Other clinical signs are conjunctivitis and reduced body weight as well as decreased egg production.

### SUMMARY

Provided herein are modified infectious laryngotracheitis viruses (ILTVs) and methods of using the same. For example, provided are attenuated ILTVs. The attenuated ILTVs can be used to illicit immune responses in avian species. Optionally, the attenuated ILTVs can be used to vaccinate an avian subject or a population of avian subjects. Optionally, an attenuated ILTV is administered in ovo to an avian egg. One or more such in ovo administrations can be used to increase the immunity of an avian herd.

An example composition comprises an attenuated infectious laryngotracheitis virus (ILTV) comprising a glycoprotein J mutation. Optionally, the mutation inhibits the expression of glycoprotein J protein. For example, the mutation can comprise a glycoprotein J promoter element mutation, wherein the mutation inhibits a function of the promoter element. Example mutations can also comprise a partial or complete deletion of a glycoprotein J nucleotide sequence such as a deletion of SEQ ID NO:1. Optionally, the mutation comprises a deletion of nucleotides 1-2291 of SEQ ID NO:1. A reporter protein expression cassette can be inserted in the deletion. The reporter protein can be green-fluorescent protein. A viral protein expression cassette can be inserted in the deletion. The viral protein cassette can be fusion protein (F) of the New Castle Disease Virus. Optionally, a glycoprotein J mutation can comprise a substitution of glycoprotein J. The substitution can, for example, comprise a rearranged ILTV sequence.

Also provided are vaccines comprising an attenuated infectious laryngotracheitis virus (ILTV) that are configured for in ovo use. For example, provided are vaccines comprising an attenuated infectious laryngotracheitis virus (ILTV) having a glycoprotein J mutation. Optionally, provided is an in ovo vaccine preparation comprising a recombinant infectious laryngotracheitis virus genome having a deletion in the glycoprotein J gene. Kits are also provided which can include an attenuated infectious laryngotracheitis virus (ILTV), means for administrating the attenuated ILTV into a hatched egg, and instructions for administration of the attenuated ILTV in ovo to an avian egg.

Methods of using modified infectious laryngotracheitis viruses include a method of preventing infectious laryngotracheitis virus (ILTV) infection in a subject or population comprising administering to one or more subjects an attenuated infectious laryngotracheitis virus (ILTV), wherein the attenuated ILTV is administered in ovo. The attenuated ILTV can optionally comprise a glycoprotein J mutation. For example, the mutation can inhibit expression of glycoprotein J protein. The mutation can also comprise a glycoprotein J promoter element mutation, wherein the mutation inhibits a function of the promoter element. Example mutations can also comprise a deletion of a glycoprotein J nucleotide sequence such as a deletion of SEQ ID NO:1. Optionally, the mutation comprises a deletion of nucleotides 1-2291 of SEQ ID NO:1. A reporter protein expression cassette can be inserted in the deletion. The reporter protein can be green-fluorescent protein. A viral protein expression cassette can be inserted in the deletion. The viral protein cassette can be fusion protein (F) of the New Castle Disease Virus. Optionally, a glycoprotein J mutation can comprise a substitution of glycoprotein J. The substitution can, for example, comprise a rearranged ILTV sequence.

Methods of eliciting an immune response in a subject include administering to the subject an attenuated infectious laryngotracheitis virus (ILTV), wherein the attenuated ILTV is administered in ovo. Also provided are methods of increasing the herd immunity of a population of avian subjects to infectious laryngotracheitis virus (ILTV), comprising administering in ovo an attenuated ILTV to one or more eggs that give rise to one or more subjects of the population. The attenuated ILTV can comprise a glycoprotein J mutation.

### DESCRIPTION OF DRAWINGS

FIGS. 1A-H are schematic illustrations of ILTV mutants. a) Schematic of the 150 kilobase (kb) ILTV genome. b) Short segment flanked by inverted repeats. Positions and direction of transcription of relevant genes are indicated. c) 7958 base pairs (bp) SphI fragment from the U_{S} region encompassing ORFs US4, US5, US6, and US7. d) Partial deletion of 2291 by of US5 encoding gJ is indicated by dotted lines. e) Structure of the gJ deletion mutant ADgJ4.1 with a GFP-expression cassette replacing the first 2291 by of US5. f) A 2188 by fragment was deleted from the 5′end of US5 to generate the gJ deletion mutant BDgJ3.2. g) Structure of BDgJ3.2. h) The 5498 by genomic fragment used for generation of the rescue mutant gJR.

FIG. 2 is a photograph showing a SDS-PAGE and Western blot illustrating baculovirus expression and purification of glycoprotein J. Coomassie blue stained SDS-PAGE (lane 1) and Western blot with an anti-RGS-6xHis MAb (lane 2) of purified gJ. Western blot of purified gJ tested with either the anti-ILTV gJ MAb; (lane 3) or a reconvalescent serum from ILTV infected chicken (lane 4). A marker (M) for the molecular weight of the proteins is shown on the left side of the figure.

FIGS. 3A-E are photographs of gels showing DNA fragments amplified by PCR from viral genomic DNA of ADgJ4.1 and BDgJ confirmed the genotype. (A) PCR amplifications on lanes 1 and 2 performed with primer pair gGupf/CMVprev and amplifications on lanes 3 and 4 performed with primer pair EGFP578fe/ClaIgIrev. Lanes 1 and 3: water control, lanes 2 and 4: ADgJ4.1 DNA, (B) PCR amplification performed with primer pair BamHIgGfw/gJ2381rev. Lanes 1 and 3: water control, lane 2: ADgJ4.1, lane 4: USDA-ch. (C) Incubation of PCR fragments from (B) with BamHI, lane 1: ADgJ4.1 and lane 2: USDA-ch. (D) PCR amplifications performed with primer pair BamHIgGfw/gJ2381rev. Lane 1: BDgJ1.1, lane 2: BDgJ 3.1, lane 3: BDgJ 3.2, and lane 4: USDA-ch. (E) PCR amplifications were performed with primer pair BamHIgGfw/gJ2381rev. Lanes 1 and 5: water control, lane 2: gJR1.3, lane 3: gJR2.4 lane 4; gJR4.3, lane 6: USDA-ch. The reaction products were analyzed on a 0.7% gel. A DNA marker is shown on each gel at the left side.

FIGS. 4A-C are photographs showing double immunofluorescence of LMH cells infected with the ILTV wildtype virus USDA-ch, the gJ deletion mutant ADgJ4.1, and the rescue mutant gJR4.3. 72 hours post infection (p.i.) cells were fixed and processed for immunofluorescence. (A) Cells infected with the wildtype virus USDA-ch (wt) show a positive signal after incubation with monoclonal antibodies (MAb's) directed either against gJ or gC. The specificity of the reaction was confirmed by using a polyclonal anti-ILTV serum from a chicken. (B) The GFP-expressing ADgJ4.1 was used to infect LMH cells. Infected cells showed a positive signal after incubation with the anti-gC MAb whereas no signal was observed after incubation with the anti-gJ MAb. The successful infection of the inspected cells was confirmed by using the anti-ILTV chicken serum. (C) Restoration of gJ expression was investigated after infection of LMH cells with the rescue mutant gJR4.3. Infected cells as indicated by the presence of gC expression did also react with a polyclonal rabbit anti-gJ serum. MAbs and polyclonal sera were diluted in all assays 1:100 and 1:500, respectively. The binding of the MAbs was visualized using goat anti-mouse Cy5-conjugated antibodies. The presence of chicken as well as rabbit antibodies was detected by using goat species-specific FITC-conjugated antibodies. The nuclei of the cells were visualized by using either propidium iodide (A and B) or 4′,6-diamidino-2-phenylindole (C). The pictures were taken using a confocal laser scanning microscope LSM 510.

FIGS. 5A-E are photographs showing Western blot analysis for the detection of gJ and gC in virions and infected cells of ILTV mutants and the wild type USDA-ch strain. (A and B) Purified virions of the gJ-deletion mutant ADgJ4.1 (lane 1) and the wildtype USDA-ch strain were tested using the anti-gJ MAb (A) and anti-gC MAb (B). (C) USDA-ch virions (lane 1 and 3) and virions of the gJ-deletion mutant ADgJ4.1 (lane 2 and 4) were incubated either with the rabbit pre-immune serum (lane 1 and 2) or the rabbit anti-gJ serum (lane 3 and 4). (D) USDA-ch virions (lane 1), uninfected CK cells (lane 2), CK cells infected with either the USDA-ch wildtype virus (lane 3) or the virus mutants gJR4.3 (lane4), BDgJ3.2 (lane 5), and ADgJ41 (lane 6) were incubated with anti-gJ MAb. (E) Same virion and CK cell preparations as shown in 5D were incubated with anti-gC MAb. Protein samples in all four gels were separated on a SDS-7.5% PAGE. The binding of the appropriate antibodies was visualized by chemiluminescence using anti-species HRP conjugated antibodies.

FIGS. 6A and B are graphs showing that virus replication but not viral entry was impaired in ILTV gJ deletion mutants. (A) Replication kinetics in CK-cells infected with USDA-ch, ADgJ4.1, BDgJ3.2, and gJR4.3 at a multiplicity of infection (m.o.i.) of 0.01. Viral titers (TCID₅₀) in supernatants were determined at 0, 24, 48, and 72 hours p.i. (B) For virus entry kinetics 500 plaque forming units (pfu) of virus were adsorbed on ice to LMH cells for 60 minutes. Temperature was shifted to 39° C. for different times (x-axis) to allow entry of adsorbed virus particles. Virus remaining on the outside of the cells were inactivated, and cells were overlaid with semisolid medium for plaque assay. Five days p.i. plaques were counted. The number of plaques at 60 minutes was set as 100% and the number of virus plaques was expressed as percent of the 60 minutes value. The percentages of entry were plotted against the entry times. The averages of three different experiments are shown. Error bars indicate standard deviations.

FIGS. 7A and B are graphs showing clinical sign scores in chickens inoculated with gJ deletion mutants ADgJ and BDgJ and subsequently challenged with USDA-ch strain. (A) Chickens were inoculated via the nasal/conjunctival route at 4 weeks of age with gJ deletion mutants ADgJ4.1 and BDgJ3.2. One control group was sham inoculated. Three weeks after inoculation, chickens were challenged with the USDA-ch strain and clinical signs were scored from days 1 to 6 post challenge. (B) Eighteen day-old SPF embryos were in ovo inoculated with gJ deletion mutants ADgJ4.1 and BDgJ3.2. One control group was sham inoculated. At 35 days of age, chickens were challenged with the USDA-ch strain and clinical signs were scored from days 1 to 7 post-challenge. For both experiments (A and B) clinical signs were scored on a scale from 1-5. The average clinical score for each day is shown on the y-axis.

FIG. 8 shows a schematic for the generation of a novel gJ deleted ILTV construct (NΔdJ ILTV).

### DETAILED DESCRIPTION

Infectious laryngotracheitis (ILT) is a viral infection of the respiratory tract of chickens, pheasants and peafowl. It can spread rapidly among birds and causes high death losses in susceptible poultry. Turkeys, ducks and geese do not get the disease, but they can spread the virus.

The etiological agent for this disease is Infectious laryngotracheitis virus (ILTV), systematically named Gallid herpesvirus 1. The main sites of ILTV replication are the larynx, trachea and conjunctiva. Severe clinical signs are observed as respiratory manifestations such as gasping, coughing, expectoration of bloody mucus, and suffocation. Other clinical signs are conjunctivitis and reduced body weight as well as decreased egg production.

ILTV has been classified as the prototype member of the genus Iltovirus of the Alphaherpesvirinae subfamily of the Herpesviridae family. In the past 50 years, the disease was mainly controlled through biosecurity and vaccination with live vaccines attenuated by consecutive passages either in chicken embryos (chicken embryo origin, CEO vaccine) or tissue culture (tissue culture origin, TCO vaccine).

The CEO vaccines, although proven to be effective to limit outbreaks in the field, possess residual virulence that can increase during passages in chickens. In the field, the unrestricted use of CEO vaccines and poor flock vaccination by coarse spray or via the drinking water has allowed vaccine strains to regain virulence, causing severe outbreaks of ILT.

More recently, the use of virus vectors such as herpesvirus of turkeys and attenuated fowlpox virus carrying glycoprotein genes of ILTV has provided a safer vaccination alternative due to their lack of transmission and no reversion to virulence. Expression of one or two ILTV genes may not, however, provide the complete immunity necessary to withstand a severe challenge. Moreover, neither of these recombinant viruses replicates in the respiratory epithelium, the primary infection site of ILTV. Mucosal immunity at the primary site of viral infection is likely to play an important role in protection from this disease.

Another strategy for the development of more effective ILTV vaccines is to engineer live-attenuated ILTV vaccines with defined deletions of non-essential genes. Viral genes coding for structural glycoproteins are targets for deletion because they are immunogenic proteins and are involved in processes of viral attachment, entry, morphogenesis, and cell- to- cell spread, consequently, their deletion is likely to result in attenuation.

In addition, an attenuated ILTV mutant lacking one or more glycoproteins can be utilized as a marker vaccine that allows the serological differentiation of infected from vaccinated animals. Deletion of genes coding for the ILTV gE and gI homologs led to non-replicating recombinant viruses, indicating that the two glycoproteins are essential for ILTV replication. However, ILTV genes encoding gC, gG, gJ, gM and gN were successfully deleted from the virus genome resulting in mutants with varied degrees of in vitro replication defects and different levels of attenuation in chickens.

Of the 12 predicted ILTV glycoproteins, only gC and gJ were recognized by ILTV specific monoclonal antibodies (MAbs). One group of MAbs recognized a 60-kDa protein that was shown to be the ILTV homologue of herpes simplex virus type 1 (HSV-1) glycoprotein C. Another group of MAbs recognized the positional homologue of HSV-1 gJ encoded by the open reading frame (ORF) 5 located within the unique short genome region of the ILTV genome and therefore designated US5.

ILTV gJ is expressed in several forms, ranging in molecular weight from 85, 115, 160, to 200 kDa from spliced and nonspliced mRNAs. During experimental infections, antibodies to glycoproteins J and C were detected earlier and in relatively higher amounts than antibodies to gB and gE. Recombinant viruses lacking gJ and gC encoding genes have been constructed indicating that these two major antibody-inducing glycoproteins are non-essential for in vitro replication of the virus.

The gC mutant in vitro replication was comparable to the wild type parental strain and to the gC rescue virus. In vivo the gC mutant virus retained some virulence, induced effective protection against disease, and significantly reduced viral shedding post-challenge. A gJ mutant constructed from the virulent ILTV strain (Fuchs et al., (2005) J. Virol. 79(2): 705-716) showed significant reduction in titers (log₁₀ 5.7 pfu/ml) when compared to the wild type virus (log₁₀ 6.5 pfu/ml) and to the gJ rescue virus (log₁₀ 6.8 pfu/ml). In chickens, the gJ mutant was significantly attenuated and induced complete protection with no shedding of the challenge virus. However, the gJ deletion mutant had to be inoculated intratracheally at a high dose in order to induce complete protection.

Provided herein are modified infectious laryngotracheitis viruses (ILTVs) and methods of using the same. For example, provided are attenuated ILTVs. The attenuated ILTVs can be used to illicit immune responses in avian species. Optionally, the attenuated ILTVs can be used to vaccinate an avian subject or a population of avian subjects. Optionally, an attenuated ILTV is administered in ovo to an avian egg that will hatch into an individual of an avian population. One or more such in ovo administrations can be used to increase the immunity of an avian herd.

The avian subject can be any avian species. For example, the subject can be a chicken, turkey, duck, goose, pheasant, quail, partridge, guinea, ostrich, emu or peafowl, as well as any other commercially processed avian and/or any avian, or an egg or eggs of the same.

An attenuated infectious laryngotracheitis virus (ILTV) can comprise a glycoprotein J mutation, wherein the mutation inhibits expression of glycoprotein J. Optionally, the mutation can inhibit the expression of glycoprotein J protein. For example, the mutation comprises a glycoprotein J promoter element mutation, wherein the mutation inhibits a function of the promoter element. Example mutations can also comprise a complete or partial deletion of a glycoprotein J nucleotide sequence such as a deletion of SEQ ID NO:1. Optionally, the mutation comprises a deletion of nucleotides 1-2291 of SEQ ID NO:1. Optionally, the mutation comprises a deletion of nucleotides 1-2188 of SEQ ID NO:1. Optionally, the mutation comprises a deletion of at least nucleotides 1-145 of SEQ ID NO:1. Optionally, the mutation does not comprise nucleotides 2185-2190 of SEQ ID NO:1. A reporter protein expression cassette can be inserted at the deletion. The reporter protein can be, for example, green-fluorescent protein. A viral protein expression cassette can be inserted in the deletion. The viral protein cassette can be fusion protein (F) of the New Castle Disease Virus. Optionally, a glycoprotein J mutation can comprise a substitution of glycoprotein J. The substitution can, for example, comprise a rearranged ILTV sequence. By rearranged ILTV sequence, it is meant that the substitution contains only ILTV sequences that have been manipulated by methods known in the art to move around portions of the genome such that the same number of nucleotides are present as a wild type, the nucleotides are therefore just arranged in a different order than a wild type ILTV sequence. This results in a lack of expression of glycoprotein J with no foreign DNA being introduced into the attenuated ILTV.

Also provided are vaccines comprising an attenuated infectious laryngotracheitis virus (ILTV), wherein the vaccine is configured for in ovo use. When in ovo administration is used, the compositions can be introduced into any region of an avian egg, including and not limited to the air cell, the albumen, the chorio-allantoic membrane, the yolk sac, the yolk, the allantois, the amnion, or directly into an embryonic bird.

Example vaccines comprise an attenuated infectious laryngotracheitis virus (ILTV) having a glycoprotein J mutation. Optionally, provided is an in ovo vaccine preparation comprising a recombinant infectious laryngotracheitis virus genome having a deletion in the glycoprotein J gene. Kits are also provided which can include an attenuated infectious laryngotracheitis virus (ILTV), means for administrating the attenuated ILTV into a hatched egg, and instructions for administration of the attenuated ILTV in ovo to an avian egg.

Methods of using modified infectious laryngotracheitis viruses include a method of preventing infectious laryngotracheitis virus (ILTV) infection in a subject or population, the method comprising administering to one or more subjects an attenuated infectious laryngotracheitis virus (ILTV), wherein the attenuated ILTV is administered in ovo. The attenuated ILTV can optionally comprise a partial or complete glycoprotein J mutation. For example, the mutation can inhibit expression of glycoprotein J protein. The mutation can also comprise a glycoprotein J promoter element mutation, wherein the mutation inhibits a function of the promoter element. Example mutations can also comprise a deletion of a glycoprotein J nucleotide sequence such as a deletion of SEQ ID NO:1. Optionally, the mutation comprises a deletion of nucleotides 1-2291 of SEQ ID NO:1. Optionally, the mutation comprises a deletion of nucleotides 1-2188 of SEQ ID NO:1. Optionally, the mutation comprises a deletion of at least nucleotides 1-145 of SEQ ID NO:1. Optionally, the mutation does not comprise nucleotides 2185-2190 of SEQ ID NO:1. A reporter protein expression cassette can be inserted at the point of the deletion. The reporter protein can be, for example, green-fluorescent protein. A viral protein expression cassette can be inserted in the deletion. The viral protein cassette can be fusion protein (F) of the New Castle Disease Virus. Optionally, a glycoprotein J mutation can comprise a substitution of glycoprotein J. The substitution can, for example, comprise a rearranged ILTV sequence.

Methods of eliciting an immune response in a subject include administering to the subject an attenuated infectious laryngotracheitis virus (ILTV), wherein the attenuated ILTV is administered in ovo. Also provided are methods of increasing the herd immunity of a population of avian subjects to infectious laryngotracheitis virus (ILTV), comprising administering in ovo an attenuated ILTV to one or more eggs that give rise to one or more subjects of the population. The attenuated ILTV can comprise a glycoprotein J mutation. Example mutations can also comprise a partial or complete deletion of a glycoprotein J nucleotide sequence such as a deletion of SEQ ID NO:1. Optionally, the mutation comprises a deletion of nucleotides 1-2291 of SEQ ID NO:1. Optionally, the mutation comprises a deletion of nucleotides 1-2188 of SEQ ID NO:1. Optionally, the mutation comprises a deletion of at least nucleotides 1-145 of SEQ ID NO:1. Optionally, the mutation does not comprise nucleotides 2185-2190 of SEQ ID NO:1.

Also provided herein are glycoprotein J deletion mutants that grow to suitable titers in CK cells and chicken embryos and induce complete protection against challenge after in ovo inoculation of 18-day-old embryonated SPF eggs.

The described compositions and vaccines can comprise a suitable carrier and an effective amount of any of the modified (e.g. recombinant) infectious laryngotracheitis virus described. The compounds and vaccines may contain either inactivated or live recombinant virus. Suitable carriers for the recombinant virus are well known in the art and include proteins, sugars, etc. One example of such a suitable carrier is a physiologically balanced culture medium containing one or more stabilizing agents such as hydrolyzed proteins, lactose, etc. An adjuvant can also be a part of the carrier of the vaccine.

A live vaccine can be created by taking tissue culture fluids and adding stabilizing agents such as stabilizing, hydrolyzed proteins. An inactivated vaccine can use tissue culture fluids directly after inactivation of the virus.

The compositions and vaccines described herein can be administered by any suitable route. For example, the compositions and vaccines can be administered in ovo, orally, parenterally (e.g., intravenously), by intramuscular injection, by intraperitoneal injection, by direct injection into an organ, transdermally, extracorporeally, topically or the like, including topical intranasal, and intratracheally. The vaccines and compositions can be applied to any organ system such as the respiratory system or the eye.

Administration or vaccination in ovo includes administering an immunogenic composition (e.g., a vaccine) to a bird egg containing a live, developing embryo by any means of penetrating the shell of the egg and introducing the immunogenic composition. Such means of administration include, but are not limited to, in ovo injection of the immunogenic composition.

Any suitable methods can be used for introducing the described compositions in ovo, including in ovo injection, high pressure spray through an egg shell, and ballistic bombardment of the egg with microparticles carrying the composition. In some examples, the described compositions can be administered by depositing an aqueous, pharmaceutically acceptable solution into avian tissue, such as muscle, which solution contains the composition to be deposited.

Where in ovo injection is used, the mechanism of injection is not critical, but it is preferred that the method not unduly damage the tissues and organs of the embryo or the extraembryonic membranes surrounding it so that the treatment will not decrease hatch rate. Suitable devices can be used for in ovo administration that can optionally comprise an injector containing a modified ILTV, with the injector positioned to inject an egg with the ILTV. In addition, if desired, a sealing apparatus operatively associated with the injection apparatus may be provided for sealing the hole in the egg after injection thereof

The appropriate volume and dosage of a composition comprising a modified ILTV to be administered can be readily determined by those skilled in the art. Therapeutic treatment, such as vaccination, involves administering to a subject a therapeutically effective amount of the compositons described herein. The terms effective amount and effective dosage are used interchangeably. The term effective amount is defined as any amount necessary to produce a desired physiologic response (e.g., partial or total protection against infectious larynogtracheitis, or eliciting an immune response in the subject). Effective amounts and schedules for administering the compositions may be determined empirically. The dosage ranges for administration are those large enough to produce the desired effect. The dosage should not be so large as to cause substantial adverse side effects. When in ovo administration is used, the dosage can be adjusted depending on factors such as egg size, with larger eggs generally receiving a larger volume and dosage versus smaller eggs. Other factors that can affect dosage or volume for in ovo or other routes of administration, include, but are not limited to, the avian species being vaccinated.

Methods of preventing infectious laryngotracheitis and vaccination methods include reducing the effects of infectious laryngotracheitis or one or more symptoms of infections laryngotracheitis (e.g., one or more respiratory symptoms, or bird-to-bird transmission of ILTV) in a bird or population of birds. Efficacy can refer to a 10%, 20%, 30%, 40%, 50%, 60%, 70%, 80%, 90%, or 100% reduction in the severity of established infectious laryngotracheitis or one or more symptom of infectious laryngotracheitis, or in the rate at which an individual or population of birds is infected with ILTV or manifests symptoms of infectious laryngotracheitis after exposure to ILTV.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention except as and to the extent that they are included in the accompanying claims.

Two gJ-negative recombinant ILTV were generated. These viruses were analyzed in vitro and in vivo in comparison to the wild type virus USDA-ch and the corresponding gJ rescue mutant. For analysis of the expression of gJ, an anti-gJ rabbit hyperimmune serum against baculovirus-expressed gJ was generated. ILTV gJ is encoded by US5 located in the short segment of the genome and was named after its positional homologue in the HSV-1 genome. The deduced amino acid sequence of ILTV US5 shares 18% identity with the respective homologous gene sORF2 of Psittacid Herpes virus 1 (PsHV-1).

ILTV and PsHV-1 are closely related and represent the two species of the genus Iltovirus within the subfamily Alphaherpesvirinae of the family Herpesviridae. ILTV gJ shares limited sequence homology with its counterpart in Equine herpesvirus (EHV)-1 and EHV-4, in which gJ is referred to as gp-2. It was shown that gp-2 plays an important role in the virulence of EHV-1. ILTV gJ has been identified as a late glycoprotein that is translated from a spliced and a non-spliced mRNA and appears as four high molecular weight proteins in SDS-PAGE. gJ is expressed in infected CK cells in four forms (approximately 85, 115, 160, and 200 kDa). gJ is dispensable for replication of the virulent strain A489 and that gJ negative mutants derived from A489 strain were able to infect chickens when inoculated intratracheally at a high dose. Chickens developed only mild signs of the disease and were protected against challenge infection. This was an important finding in the light of the observation that gJ represents a major antibody-inducing antigen of ILTV.

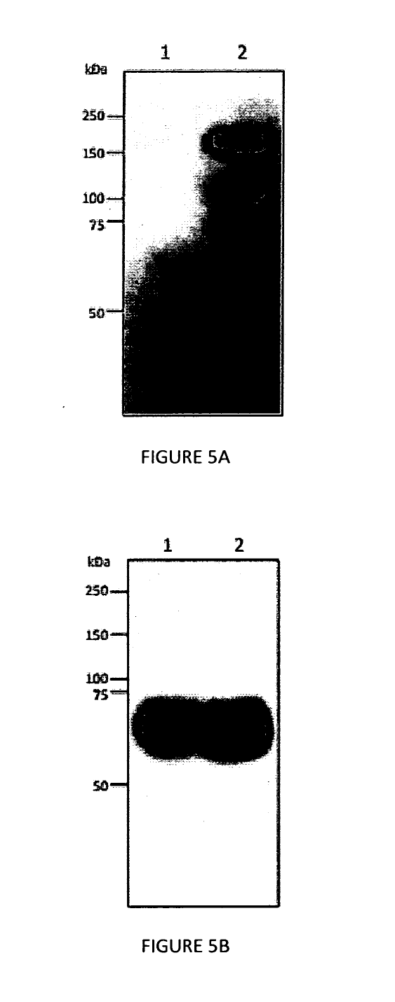

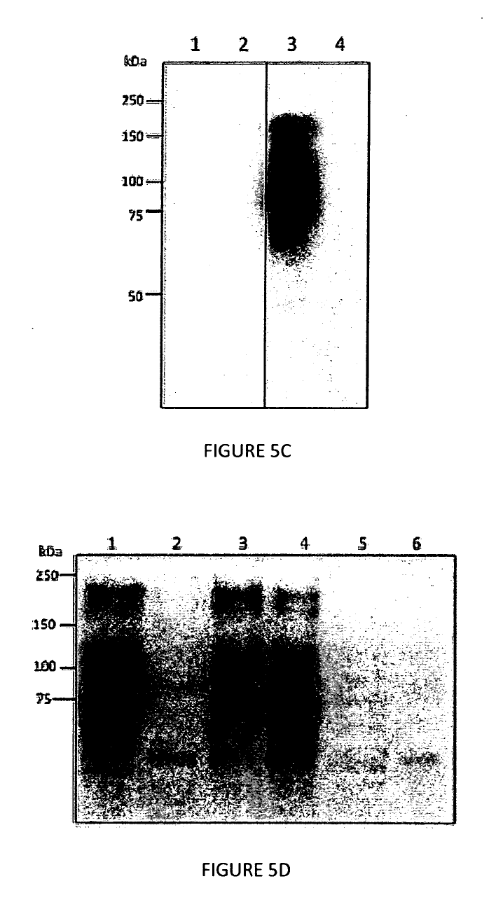

ILTV gJ was targeted for the development of a gene deletion marker vaccine against ILT. Two gJ deletion mutants were generated, one expressing the green fluorescent protein under the control of the CMV immediate early promoter, the other void of any foreign DNA insertions. A gJ rescue mutant with a reconstituted gJ gene was also generated as a control for the recombinant virus mutants. Partial deletion of US5 resulted in complete abolishment of gJ expression in both deletion mutants (ADgJ4.1 and BDgJ 3.2) and reintroduction of wt US5 into the genome of mutant ADgJ4.1 reconstituted gJ expression in the rescue mutant gJR4.3. Absence of reactivity in immunofluorescence assays and Western blots of both the anti-gJ MAb and the rabbit anti-gJ serum, ruled out expression of truncated forms of gJ from the 668 by remnant of US5 that was retained in these mutants. Expression of gC was assayed as a control to monitor the presence of expression of viral glycoproteins other than gJ in the mutant viruses. As observed in FIG. 5E the gC expressed from either mutant ADgJ4.1 and BDgJ3.2 (FIG. 5E, lanes 5 and 6) showed a slightly decreased electrophoretic mobility as compared to gC expressed by gJR4.3 and USDA-ch infected cells (FIG. 5E, lanes 3 and 4). As gJR4.3 was derived from ADgJ4.1 and gC is encoded by UL44 located on the large segment of the genome, it is unlikely that the altered mobility of gC in the gJ deletion mutants was caused during the homologous recombination event in the unique short segment of the genome.

Replication kinetics experiments indicated that the gJ deletion mutants ADgJ4.1 and BDgJ3.2 replicated less efficiently than the viruses expressing gJ (USDA-ch strain, rescue mutant gJR4.3). Infectious virus was detected in supernatants of cells infected with the gJ deletion mutants at 100-fold lower titers than the parental virus USDA-ch and the rescue mutant gJR at all times tested during the replication kinetics experiments. As compared to the gJ rescue mutant, gJ deletion mutants did not show impairment in cell entry kinetics, indicating that glycoprotein J does not play a role in viral entry of ILTV. gJ deletion mutants ADgJ and BDgJ were not impaired in their ability to spread from cell to cell since the average plaque sizes of the gJ deleted mutants were not smaller than the average plaque sizes of the USDA-ch or gJR viruses.

Similar to the replication of ADgJ4.1 and BDgJ3.2 in cell culture, replication of gJ deletion mutants in embryonated chicken eggs was impaired as compared to the USDA-ch and the rescue mutant gJR4.3. While gJR4.3 reached 10 to 50 fold higher titers than in CK-cells, replication of ADgJ4.1 was initially less efficient but improved after four passages reaching then only tenfold lower titers (10⁶ TCID₅₀/ml) than the rescue mutant gJR4.3 (10⁷ TCID₅₀/ml). On the other hand, replication of BDgJ3.2 in CAMs was very inefficient.

When administered via the conjunctival/nasal route to 3 week old chickens, the gJ deletion mutants showed strong attenuation as indicated by the absence of viral DNA in conjunctiva and trachea. Neither gJ deletion mutant impaired hatchability when compared to the sham-inoculated control, in spite of efficient replication in chicken embryos of ADgJ4.1. Both mutants were capable of protecting against disease when administered in ovo as indicated by a significant reduction in clinical signs after a severe ILTV challenge.

### Example 1

Generation of Infectious laryngotracheitis virus (ILTV) glycoprotein J deletion mutants: In vitro growth characteristics and protection efficiency after in ovo administration.

### Materials and Methods

Cells and viruses. Primary chicken kidney (CK) cells were prepared as previously described (Tripathy (1998), A Laboratory Manual for the Isolation and Identification of Avian Pathogens, 4^{th} ed.) and used for propagation of virus and determination of titers as tissue culture infectious dose 50 (TCID₅₀). The chicken liver tumor cell line LMH (Kawaguchi et al., (1987) Cancer Research, 47, 4460-4464) was cultivated in Dulbecco's Minimal Essential Medium (DMEM) supplemented with 10% fetal bovine serum and used for transfection and plaque purification.

Infected or transfected LMH cells were incubated in DMEM containing 2% FBS and antibiotic/antimycotic (Invitrogen, Carlsbad, Calif., USA). Cells were incubated in a humidified incubator at 39° C./ 5%CO₂. Virus strains used were the USDA reference strain (USDA-ch) and field isolate 63140/C/08/BR previously characterized as genotype V (Oldoni et al., (2008) Avian Dis. 52:59-63). The Spodoptera frugiperda ovary cell line Sf-9 was used for generation of recombinant baculovirus as well as for production of the recombinant proteins. Sf-9 cells were cultivated in HyClone SFX® medium (Fisher, Pittsburg, Pa.) containing penicillin and streptomycin at 28° C.

Generation and purification of recombinant glycoprotein J. All the oligonucleotides used were synthesized by Integrated DNA technologies (IDT, Coralville, Iowa, USA) and are listed in Table 1.

TABLE 1
Primers

| Name | Sequence | RE^{A} Sites |
|---|---|---|
| gJfw | 5′-AGCGGATCCATGGGGACAATGTTAGTGTTGC-3′^{B} (SEQ ID NO: 2) | BamHI |
| gJrev | 5′-GTGCGGCCGCCTAatggtgatggtgatggtgacttcctctAAAATAAATGGC | NotI |
| | GGTCCATAGCG-3′ (SEQ ID NO: 3) | |
| gJdel5′FW | 5′-GCAGAATTCAGTTGCGCTGAGTACCG-3′ (SEQ ID NO: 4) | EcoRI |
| gJdel5′REV | 5′-CGTCCCGGGCGAAATACGCTGCACGCC-3′ (SEQ ID NO: 5) | XmaI |
| gJdel3′FW | 5′-CGAGCATGCATCTCCCTATAGGGTAGAAAC-3′ (SEQ ID NO: 6) | SphI |
| gJdel3′REV | 5′-CCTAAGCTTATGAGCGTGAGGCGTGGC-3′ (SEQ ID NO: 7) | HindIII |
| EGFPexFW | 5′-GCACCCGGGCCAGATATACGCGTTGAC-3′ (SEQ ID NO: 8) | XmaI |
| EGFPexREV | 5′-CGTCATAGAGCCCACCGCATCC-3′ (SEQ ID NO: 9) | — |
| gJrescFW | 5′-GCTACCCGGGCTTCAGTTGCGCTGAG-3′ (SEQ ID NO: 10) | XmaI |
| gJrescREV | 5′-CGATCCCGGGCGTGGCATGTAGGAAGAAACC-3′ (SEQ ID NO: 11) | XmaI |
| gGrev | 5′-GCTGAATTCCTCGGCGAAATACGCTGCACG-3′ (SEQ ID NO: 12) | EcoRI |
| gDpfw | 5′-GCAGAATTCCATGAGATGTCGACG-3′ (SEQ ID NO: 13) | EcoRI |
| UL48fw | 5′-CACGGATCCATGGAAGAAGAATCTTCC-3′ (SEQ ID NO: 14) | BamHI |
| UL48rev | 5′-GCTGCGGCCGCTTAGGGCATAGGTGTATCAAGG-3′ (SEQ ID NO: 15) | NotI |
| gGup fw | 5′-GTCTTCACTCGATATCATGG-3′ (SEQ ID NO: 16) | — |
| CMVp rev | 5′-GTCATTATTGACGTCAATGG-3′ (SEQ ID NO: 17) | — |
| EGFP578fw | 5′-CGTGCTGCTGCCCGACAACC-3′ (SEQ ID NO: 18) | — |
| ClaI-gI rev | 5′-CAGAAGACGATCGATGAGTGC-3′ (SEQ ID NO: 19) | ClaI |
| BamHI-gGfw | 5′-GGCAATGGATCCCTGGTGC-3′ (SEQ ID NO: 20) | BamHI |
| gJ2381 rev | 5′-CTGTTCCCAGAAATTTCATCC-3′ (SEQ ID NO: 21) | — |
| gJ1932fw | 5′-CGAACCTGTGCCTTTCACCCG-3′ (SEQ ID NO: 22) | — |
| M13 (−47) | 5′-CGCCAGGGTTTTCCCAGTCACGA-3′ (SEQ ID NO: 23) | — |
| M13 rev | 5′-CACACAGGAAACAGCTATGACCAT-3′ (SEQ ID NO: 24) | — |

^{A}Restriction enzymes used for the cloning procedure.
^{B}Restriction enzyme cleavage sequences are underlined.
The sequence encoding for the the RGS-6xHis sequence is shown in lower case. Start and stop codons for the gJ ORF are printed in bold.

The open reading frame (ORF) US5 encoding gJ (SEQ ID NO:1) was amplified from purified viral DNA of ILTV 63140 by high fidelity PCR using Pfx polymerase (Invitrogen, Carlsbad, Calif.) and primers gJfw (SEQ ID NO:2) / gJrev (SEQ ID NO:3) (Table 1).

The ILTV US5 open reading frame (encoding glycoprotein J) is SEQ ID NO:1:

The PCR product encoding a C-terminally located RGS-6xHis tag sequence was cloned into the eukaryotic expression vector pcDNA3® (Invitrogen, Carlsbad, Calif.) and in the baculovirus transfer vector pFastBacDual® (Invitrogen, Carlsbad, Calif.). Recombinant baculovirus was generated using the Bac-to-Bac® system (Invitrogen, Carlsbad, Calif.) according to the manufacturer's recommendations. Briefly, recombinant pFastBacDual® plasmid DNA was transformed in E.coli DH10Bac (Invitrogen, Carlsbad, Calif.) that contain a shuttle vector and a helper plasmid necessary for the transposition of the expression cassette from pFastBacDual® (Invitrogen, Carlsbad, Calif.) into the baculovirus bacmid DNA.

Recombinant baculovirus bacmids were selected as recommended by the manufacturer and identified by PCR using GoTaq® polymerase (Promega, Madison, Wis.). Once selected, recombinant baculovirus bacmid DNA was transfected in Sf-9 cells using Mims TransIT-Insecta transfection reagent (Roche, Basel, SE).

Recombinant baculovirus was rescued, plaque purified, and analyzed by indirect immunofluorescence assay (IFA) and Western blot using a monoclonal antibody to RGS-6xHis (Qiagen, Hilden, DE) and a FITC conjugated anti-mouse antibody (SIGMA, St. Louis, Mo.). For propagation of the recombinant baculovirus, Sf-9 cells were grown in shaker cultures and infected at a multiplicity of infection (m.o.i.) of 1 and a cell density of 4×10⁶ cells/ml for 72 hours at 28° C. Glycoprotein J of ILTV was purified from infected Sf-9 cultures by immobilized metal affinity chromatography (IMAC) using the Talon® kit (Clontech, Mountain View, Calif.) according to the instructions provided by the manufacturer. Briefly, cells were sedimented by centrifugation at 3000 rpm, 4° C. for 15 minutes, the supernatant was discarded, and the cell pellet was resuspended in lysis buffer containing 2% (w/v) Igepal 630 (SIGMA, St. Louis, Mo.) in equilibration buffer (pH 8.0, Talon® kit) containing lx complete protease inhibitors (Roche, Basel, SE). After a 15 minute incubation on ice, the lysate was centrifuged as described above. Prewashed Talon® resin was added to the supernatant and incubated on a rocker platform for 1 hour at room temperature. Washing and elution of His-tagged proteins was performed as recommended by the manufacturer. Protein concentration of the eluted protein was determined using the Micro BCA Protein Assay Kit (Pierce, Waltham, Mass.). Identity and purity of the preparations were analyzed by SDS-PAGE and Western blot.

Generation of recombinant glycoprotein J hyperimmune serum. Hyperimmune serum was produced in the Polyclonal Antibody Facility unit at the University of Georgia (Athens, Ga.). Briefly, a New Zealand white rabbit (SPF) was injected with 400 μg of purified glycoprotein J resuspended in 500 ul PBS and an equal volume of complete Freund's adjuvant. Booster injections were done with incomplete Freund's adjuvant. The rabbit was exsanguinated after two booster injections and the serum was stored at −20° C.

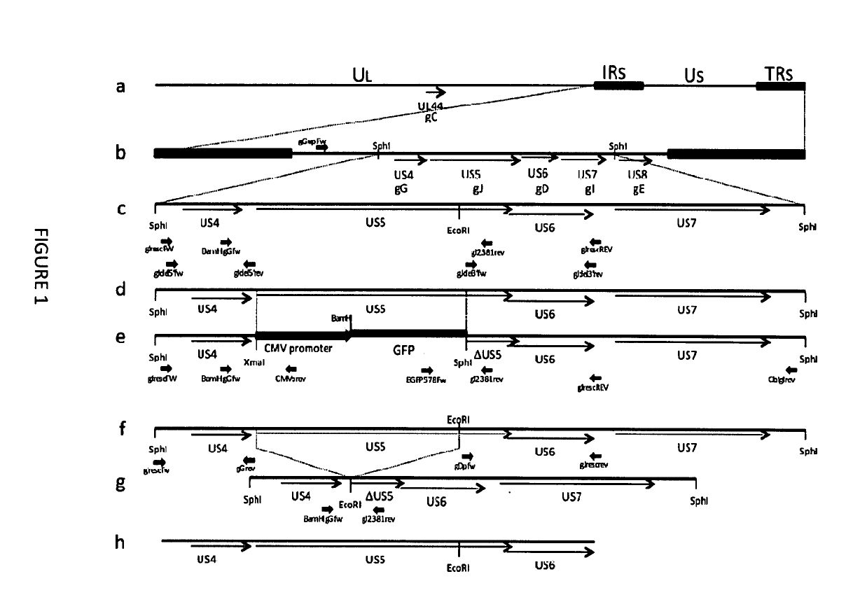

Construction of homologous recombination and expression plasmids. In order to inactivate the expression of glycoprotein J (gJ) viral DNA fragments were amplified by high fidelity PCR using Pfx polymerase (Invitrogen, Carlsbad, Calif., USA). Primers gJdel5′FW/gJdel5′REV (Table 1, FIG. 1c) containing EcoRI and XmaI restriction enzyme (RE) cleavage sites, respectively, were used to amplify a 1375 by fragment located upstream of US5. Since the 3′-coding region of gJ partially overlaps with the downstream coding region US6 of glycoprotein D (gD), the coding sequence of gJ was not entirely deleted to avoid the inactivation of gD expression (FIG. 1).

Primers gJdel3′FW/gJdel3′REV (Table 1, FIG. 1c) containing SphI and HindIII RE cleavage sites were used to amplify a 1844 by fragment containing 658 by from the 3′end of US5 (2958 bp) and 1191 by from the 5′end of US6 (1305bp). The EGFP ORF was excised from the plasmid pEGFP1 (Clontech, Mountain View, Calif., USA) by restriction enzyme digestion with BamHI and NotI and subcloned into appropriately digested pcDNA3 (Invitrogen, Carlsbad, Calif., USA) to obtain pcEGFP. The functionality of pcEGFP was tested by transient transfection in LMH cells and fluorescence microscopy.

The EGFP expression cassette consisting of the CMV promoter, the EGFP ORF and the bovine growth hormone polyadenylation signal sequence was amplified with Pfx polymerase using primers EGFPexFW/EGFPexREV containing a XmaI RE cleavage site (Table 1). The 1375 by 5′ PCR product was cleaved with EcoRI and XmaI, the EGFP expression cassette was cleaved with XmaI and SphI and the 1844 by 3′ end PCR product was cleaved with SphI and HindIII. Fragments were gel purified and ligated into EcoRI/HindIII cleaved pUC19 DNA. The recombinant plasmid pU-EGFPdeltagJ containing this combined insert of 4898 by was cloned and analyzed by restriction digestion, transient transfection and fluorescence microscopy.

For generation of the gJ rescue mutant a 5483 by fragment of the US region encompassing US4, US5 and US6 (FIG. 1) was amplified by high fidelity PCR using Pfx polymerase (Invitrogen, Carlsbad, Calif.) and primers gJrescFW and gJrescREV (Table 1, FIG. 1c) from viral DNA and cloned in XmaI cleaved pUC19 after restriction digestion with XmaI and agarose gel purification. Recombinant plasmid was analyzed by restriction enzyme analysis and the sequence was confirmed (pU-gJresc).

For generation of a gJ deletion mutant void of inserted foreign DNA sequences a 1384 by fragment upstream US5 including US4 was amplified by high fidelity PCR using gJrescFW and gGrev primers (Table 1, FIG. 1f) with XmaI and EcoRI RE cleavage sites, respectively. Secondly a 1937 by fragment encompassing the 3′ portion of US5 and partial US6 was amplified using primers gDpfw and gJrescREV (Table 1, FIG. 1f) containing EcoRI and XmaI.

The two PCR fragments were cleaved with XmaI and EcoRI and ligated to XmaI cleaved pUC19 and transformed in E.coli NEB5alpha (New England Biolabs, Ipswich, Mass.). Recombinant plasmids were analyzed by restriction digestion and sequencing and one plasmid (pU-deltagJ) was selected.

For use as a helper protein in virus rescue after co-transfection with virus DNA, the open reading frame (ORF) encoding the UL48 homolog of ILTV was amplified from purified viral

DNA by high fidelity PCR using primers UL48fw and UL48rev (Table 1) specifying BamHI and NotI restriction sites, respectively. The 1211 by PCR product was incubated with BamHI and NotI and cloned in appropriately cleaved pcDNA3. Recombinant plasmid pcUL48 was analyzed by restriction digestion and sequencing. A eukaryotic expression plasmid encoding ILTV ICP4 (pRcICP4) was used.

Generation of ILTV gJ deletion mutants. Virions from the USDA challenge strain (USDA-ch) were sedimented from supernatants of infected CK-cell cultures by centrifugation at 82667x g, 4° C., for 1 hour. Viral DNA was prepared by standard phenol-chloroform extraction method and analyzed by restriction enzyme digestion using EcoRI. LMH cells were cotransfected with 1, 2, 3, 4, 5, or 6 μg viral DNA, 0.5 μg of each of the helper plasmids (pRcICP4, pcUL48), and 1 μg of the recombination plasmid (either pU-EGFPdelta gJ, pU-gJresc, or pU-deltagJ) using the Mims® mRNA transfection kit (Roche, Basel, SE).

Transfected cultures showing cytopathic effect (CPE) were scraped into the medium and used to infect LMH cells at different dilutions. Cultures were inspected using an inverted fluorescence microscope (Axiovert® 40 CFL, Carl Zeiss MicroImaging, Inc. Thornwood, N.Y., USA) and green fluorescent plaques were aspirated under visual control using a 100 μl pipette. Picked plaques were resuspended in 100 μl DMEM/2%FBS and used for infection of LMH cells. Plaque purifications were repeated until no plaques without fluorescence were observed in two subsequent passages. DNA for analysis by PCR was prepared from infected cell cultures using the QiAmp® DNA Blood mini kit (Qiagen, Hilden, Del.).

Indirect immunofluorescence assay. Double immunofluorescence of infected LMH cells was performed with monoclonal antibodies, chicken and/or rabbit sera to confirm the absence of gJ expression by the deletion mutants and reconstitution of gJ expression in the rescue mutant. Briefly, LMH cells were seeded in chamber slides and infected with USDA-ch, ADgJ4.1, and gJR4.3 at a multiplicity of infection (m.o.i.) of 0.05. At 72 hours p.i. cells were fixed with ice-cold ethanol and processed for immunofluorescence using monoclonal antibodies specific for gJ (mab 25-5) or gC (mab 28-5).

Reconvalescent sera from ILTV-infected chickens or gJ rabbit hyperimmune serum were used as second species antibodies to perform double immunofluorescence. The anti gJ and gC MAbs were diluted 1:100 and the polyclonal sera (ILTV chicken reconvalescent serum, gJ rabbit hyperimmune serum) were diluted 1:200 in phosphate buffered saline (PBS).

After incubation with the primary antibodies, cells were washed with PBS three times and the binding of MAbs was visualized using a secondary goat anti mouse Cy5-conjugated antibody while bound chicken and/or rabbit antibodies were visualized by incubation with a goat anti-species FITC-conjugated antibodies (SIGMA). Secondary anti-species antibodies were diluted in PBS containing 0.001% Evan's blue and incubated for 1 hour. Cells were washed and briefly rinsed with distilled water prior to air-drying and mounting in 2.5% 1,4 diazabicyclo [2.2.2] octane (DABCO)/90% glycerol. Slides were inspected either by conventional fluorescence microscopy using an Axiovert ® 40 CFL fluorescence microscope (Carl Zeiss MicroImaging GmbH, Jena, Germany) or by confocal LASER scanning fluorescence microscopy using a confocal LASER scan microscope LSM 510 (Carl Zeiss MicroImaging GmbH, Jena, Germany).

Western blot. CK cells were infected with the USDA-ch strain or ADgJ4.1 mutant at a m.o.i. of 0.01. Three to 4 days p.i., when the majority of the cells were detached, the medium was clarified from cell debris by low speed centrifugation (2000 x g, 10 minutes, 4° C.). Virions were sedimented from the supernatants by ultracentrifugation at 82667x g, 4° C. for 60 minutes. The protein concentration was determined using the Micro BCA Protein Kit (Pierce, Waltham, Mass.). The sediment was lysed in 20 mM Tris-Cl; pH7.4, 1 mM EDTA, 150mM NaCl containing 1x complete protease inhibitor (Roche, Basel, SE) and 0.5 vol 3% N-laurylsarcosinate, 75 mM Tris-Cl pH 8.0, 25 mM EDTA. Thirty μg of protein were loaded per lane and separated under reducing conditions by SDS-10% polyacrylamide gel electrophoresis (SDS-PAGE) and transferred to nitrocellulose membranes by semi-dry electrotransfer in 25 mM Tris, 150 mM glycine, 10% methanol using a Transblot SD transfer cell (BioRad, Hercules, Calif., USA) at 22 V for 80 minutes.

Membranes were blocked in 5% dried skim milk in TBS-T (3.0 g Tris, 8.8 g NaCl, 0.2 g KCl per L, pH7.4) overnight at 4° C. MAbs were diluted 1:500 and rabbit anti-gJ hyperimmune serum was diluted 1:1000 in TBS-T and incubated for 1 hour at room temperature. After incubation membranes were washed three times for 10 minutes with TBS-T and blocked again in 5% dried milk in TBS-T prior to incubation with horseradish peroxidase (HRP)-conjugated anti-species antibodies (SIGMA) diluted in TBS-T for 2 hours at RT.

After washing three times in TBS-T immobilized HRP was detected by chemoluminescence using the Immobilon Western HRP substrate (Millipore, Billerica, Mass., USA). Chemoluminescence was visualized using the Kodak Gel Logic Imaging System (Carestream Health, New Haven, Conn., USA). The absence of gJ in mutant-infected cells as well as reconstitution of gJ expression in the rescue mutant infected cells was also tested by Western blot. Briefly, CK cells were infected at an m.o.i. of 0.1 with USDA-ch, gJR4.3, BDgJ3.2, and ADgJ4.1. Infected cells were lysed in SDS-PAGE sample buffer 48 hours p.i. Similar amounts were separated by SDS-PAGE (10% polyacrylamide for gC and 7.5% for gJ) and electrotransferred to nitrocellulose membranes. Blots were probed either with the rabbit anti-gJ hyperimmune serum or with gJ and gC-specific MAbs.

Replication and entry kinetics of mutants and wild type viruses. Two-step replication kinetics was performed to determine if the absence of gJ expression influenced the in vitro replication of the viral mutants. Briefly, CK cells were infected with USDA-ch, ADgJ4.1, BDgJ3.2, and gJR4.3 at an m.o.i. of 0.01. After adsorption for 60 minutes at 39° C., the inocula were removed from the cells, washed with medium and cells were overlaid with DMEM/2%FBS/antibiotic and incubated at 39° C. At 0, 24, 48, and 72 hours post-infection supernatants were collected and virus titers were determined as TCID₅₀ on CK cells. To evaluate if there was a defect in viral entry for gJ deleted mutants as compared to the USDA-ch and the gJ rescue mutant 500 plaque forming units (pfu) of each of the three mutant viruses and the parental virus USDA-ch were adsorbed to LMH cells in 6-well plates for 60 minutes on ice. The inocula were removed and cells were overlaid with warm medium and incubated at 39° C. At 5, 10, 20, 40, and 60 minutes extracellular virus was inactivated by incubation with citrate buffer (40 mM citric acid, 10mM KCl, 135 mM NaCl) pH 3.0 for 1 minute at room temperature, washed once with DMEM/2%FBS/antibiotic and finally overlaid with MEM containing 0.5% methylcellulose, 2%FBS and antibiotic. 5 days p.i. cells were fixed with 5% formaldehyde in PBS and stained with 1% crystal violet in 50% ethanol. Plaques were counted, numbers at different times were expressed as percentage of the respective plaque numbers at 60 minutes and plotted against the different times.

Propagation of mutants and wild type virus in chicken embryos. In order to assess replication of the gJ mutants ADgJ4.1 and BDgJ3.2 in embryonated eggs, the CAMs of 9-day old chicken embryos were inoculated with 100 μl of ADgJ4.1 and BDgJ3.2 containing 10⁴ TCID₅₀, respectively. Eggs were incubated at 37° C. for 5 days, CAMs were collected and homogenized in cell culture medium using the FastPrep® system (MPbiomedical, Solon, Ohio). Titers in the supernatant were determined as TCID₅₀ on primary CK cells. Remainders of the supernatants were used for serial passages on CAMs of 9-day-old chicken embryos as described above.

Conjunctival/nasal inoculation of mutants and challenge experiment. Four-week-old SPF white leghorn chickens were inoculated via the conjunctival/nasal route with 10⁴ TCID₅₀ of the mutants ADgJ4.1 and BDgJ3.2 and the USDA wild type strain (USDA-ch) to evaluate the virulence of the virus mutants. One group of hatchmates was sham-inoculated with cell culture medium as control. Clinical signs were monitored daily and scored as previously described on a scale of 0-5 (Oldoni et al., (2009) Avian Dis. 52:59-63) from day 1 to 6 after inoculation. To determine if chickens inoculated with ILTV mutants were protected against a challenge infection, three weeks after inoculation, infected and control chickens were challenged with 10⁴ TCID₅₀ of the USDA strain per bird delivered via the conjunctival and nasal route. Clinical signs were monitored up to six days after challenge and scored as described above.

Quantitative PCR. Swabs were collected four days after the first inoculation and processed using the QiAmp® DNA Blood Mini kit (Qiagen, Hilden, Del.). Viral genome copies were quantified by real-time PCR as described (Callison et al., (2007) Avian Dis. 50:50-54).

In ovo inoculation of mutants and challenge experiment. For the determination of the level of attenuation of the gJ mutants in embryonated eggs twenty-five 18-day-old SPF embryos (Sunrise Farms, Catskill, N.Y., USA) were inoculated either with ADgJ4.1 or BDgJ3.2. A third group of embryos was sham inoculated with sterile cell culture medium. The inoculated eggs were further incubated and hatchability was determined. At 35 days after hatch chickens were challenged with the USDA-ch strain at a dose of 10⁵ TCID₅₀ per chicken inoculated by the conjunctival and nasal route. Clinical signs were scored on a scale from 0 to 5 as previously described by Oldoni et al., (2009) Avian Dis. 52:59-63 up to 7 days after challenge.

### Results

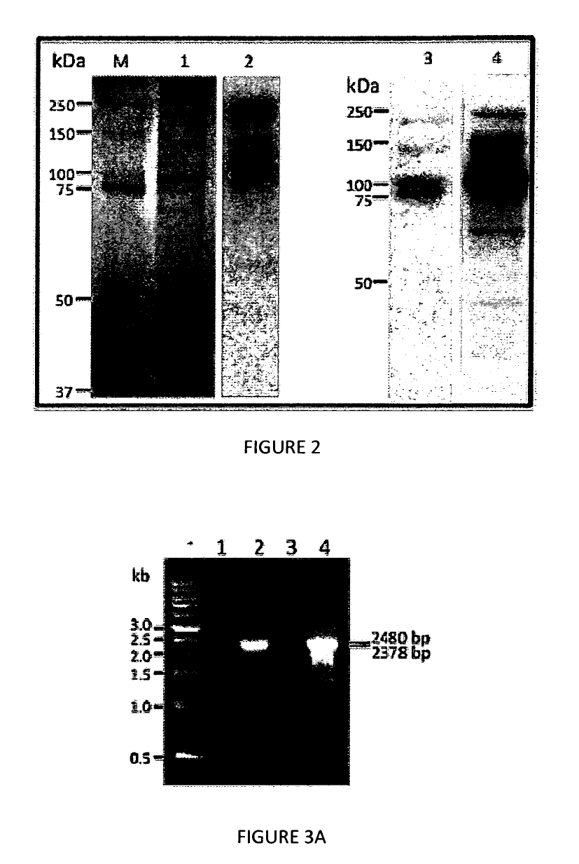

Expression of recombinant glycoprotein J. Purified gJ expressed from a recombinant baculovirus in Sf-9 cells was separated by SDS-7.5%PAGE and analyzed by Coomassie blue staining and Western blot (FIG. 2). Multiple high molecular weight proteins were observed in the Coomassie blue stained gel (FIG. 2, lane 1). In a parallel Western blot, the anti-RGS-6xHis MAb bound to similar proteins confirming the presence of the RGS-6xHis sequence (lane 2). An anti-gJ MAb (lane 3) and reconvalescent sera from ILTV infected chickens (lane 4) both bound to four proteins of approximately 80, 100, 140, and 180 kDa. In addition, the chicken serum recognized proteins at approximately 60 kDa and 40 kDa. Although the recombinant glycoprotein J was significantly enriched during the purification process, cellular proteins were still detected by Coomassie staining of the gels (lane 1). However, three immunizations with the recombinant gJ protein preparation resulted in a specific rabbit hyperimmune serum which was used in immunofluorescence and Western blot for the characterization of the gJ deletion mutants (FIG. 2).

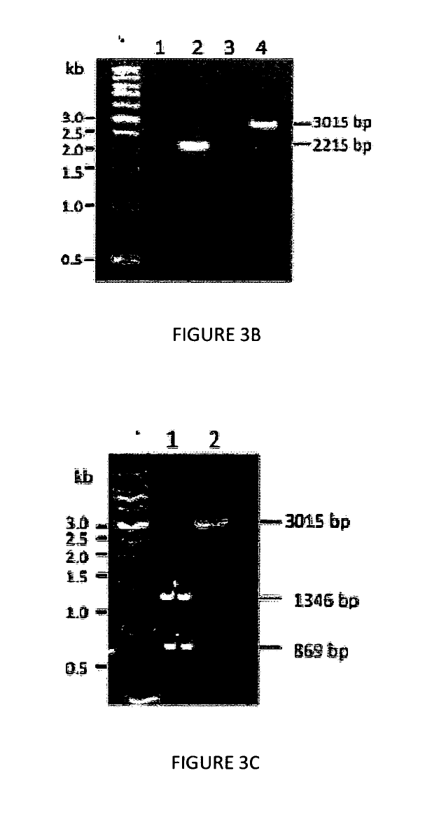

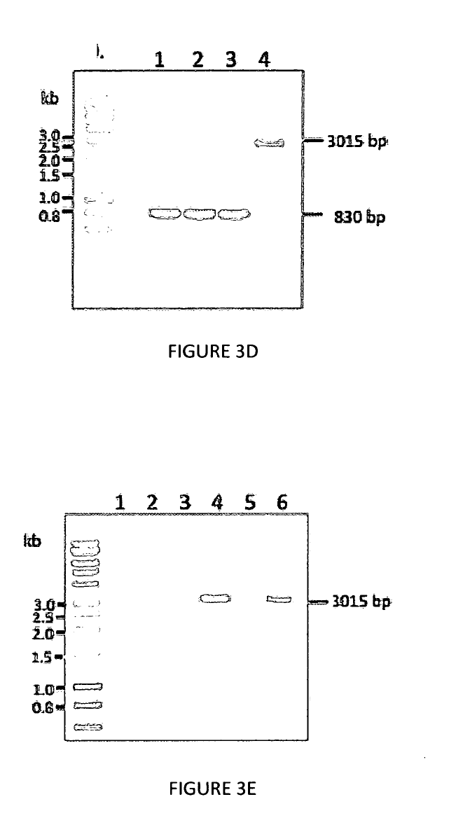

Generation of gJ deletion mutants. DNA from the USDA challenge strain (USDA-ch) was co-transfected with plasmid pU-EGFPdeltagJ, and the helper plasmids pRcICP4 and pcUL48. Five days after transfection, several individual plaques showing green fluorescence were isolated using the inverted fluorescence microscope. Three plaques were sequentially plaque purified twice in LMH cells. Only plaques that produced a progeny of only green fluoresecence plaques were selected and subsequently propagated. Viral DNA from cells infected with the EGFP encoding clone ADgJ4.1 was analyzed by PCR to confirm the accuracy of the homologous recombination (FIG. 3). ILTV wildtype DNA was used as control.

A reverse primer binding to the CMV (CMVprev) promoter and the forward primer (gGupfw) complementary to a sequence located upstream of the gG orf US4 produced the expected product of 2378 by (FIG. 1 and FIG. 3A), a forward primer complementary to the EGFP ORF (EGFP578fw) and a reverse primer (ClaIgIrev) complementary to the downstream region of US7 (FIG. 1 and FIG. 3A) amplified a 2480 by product as expected. This result indicated that the insertion of the EGFP expression cassette occurred at the target site of the genome.

To verify the lack of wild type virus DNA in the mutant virus ADgJ4.1 preparation, PCR analysis was performed with primers that bind to both viral genomes but outside of the recombination sequence. The BamHIgGfw and gJ2381rev primers (FIG. 1, Table 1) amplified a 3015 by and a 2215 by fragment when USDA-ch and ADgJ4.1 DNA were used as templates, respectively (FIG. 3B). This result showed that a shorter DNA sequence was present at the target site as indicated by the shorter PCR fragment. To verify the identity of the target insert both PCR fragments were digested with BamHI. The 2215 by PCR product obtained from ADgJ4.1 was cleaved at the BamHI site located between the CMV promoter and the EGFP ORF resulting in two fragments of 1346 and 869 bp, whereas the PCR product amplified from USDA-ch DNA remained intact (FIG. 3C).

Primers used for PCR were also used to partially sequence the PCR product from ADgJ4.1 and the wildtype DNA in order to confirm the sequences of the mutant and wildtype DNA. To generate an ILTV mutant with an inactivated gJ gene not carrying any foreign DNA, the EGFP expressing gJ deletion mutant ADgJ4.1 was propagated on CK cells, virions were purified and viral genomic DNA was prepared. After co-transfection of LMH cells with the helper plasmids and the recombination plasmid pU-deltagJ, several non-fluorescent plaques were isolated, plaque purified and propagated. In this case, the selection criterion was the absence of green fluorescence. Three of these virus plaques were purified and designated as BDgJ1.1, BDgJ3.1, or BDgJ3.2. Viral DNA from the BDgJ clones was prepared and analyzed by PCR. Primers BamHIgGfw and gJ2381rev produced 830 by fragments with BDgJ viral DNA and a 3015 by fragment with USDA-ch wildtype DNA (FIG. 1G and FIG. 3D). Sequences obtained from both DNA fragments using the PCR primers confirmed the identity of both fragments.

Next, a rescue mutant was generated where gJ expression was restored. Viral DNA from ADgJ4.1 was used for co-transfection with pU-gJresc and helper plasmids. Recombination of mutant viral DNA with the insert of pU-gJresc was expected to repair the mutated section within the U_{S} segment and fully restore US5, resulting in a mutant identical to the wild type virus. Again non-fluorescent plaques were picked and virus was plaque purified and propagated.

Viral DNA from three of plaques designated as gJR1.3, gJR2.4, or gJR4.3 was prepared and analyzed by PCR. Amplifications using primers BamHIgGfw and gJ2381rev produced 3015 by fragments from gJR clone 4.3 and USDA-ch DNA as expected (FIG. 1G and FIG. 3E) indicating correct insertion from the recombinant plasmid pU-gJresc. No PCR product was obtained from DNA from clones 1.3 and 2.4, and consequently these viruses were discarded.

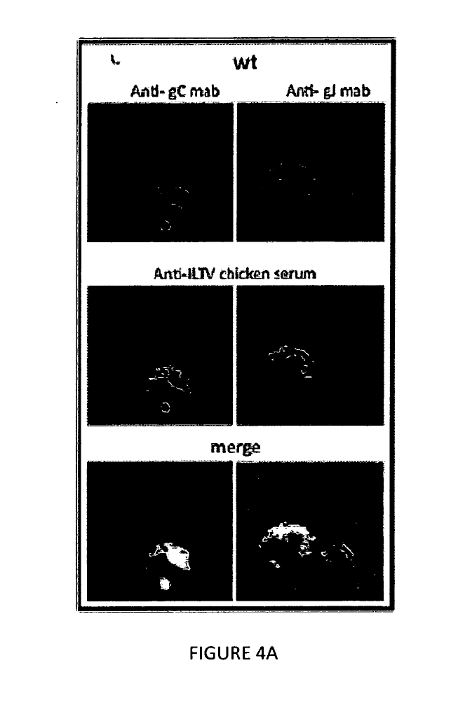

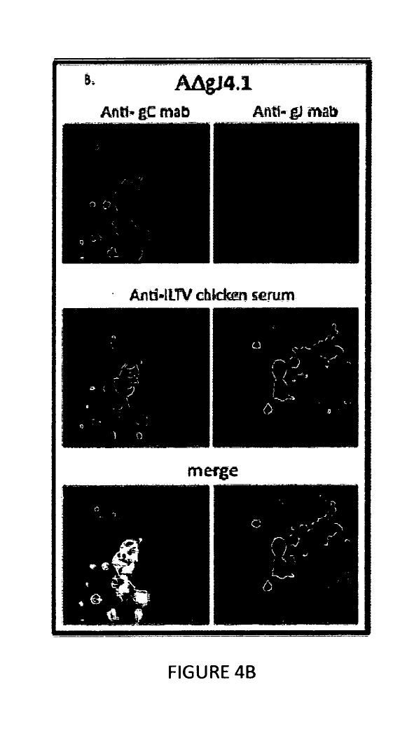

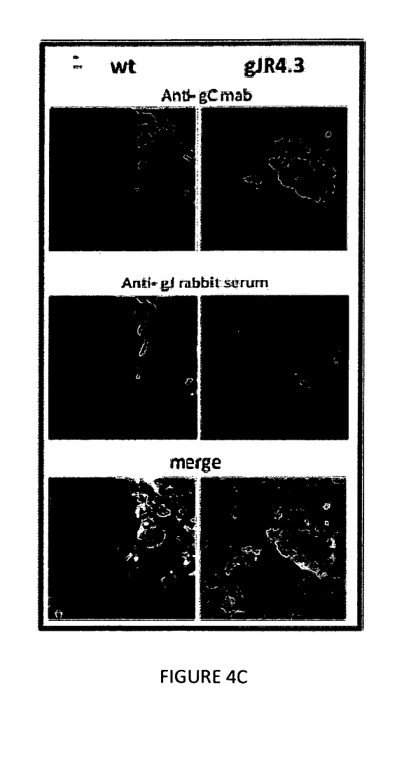

Partial deletion of US5 abolishes gJ expression. The lack of gJ expression by the ADgJ4.1 mutant and reconstitution of gJ expression in the rescue mutant gJR4.3 were assayed by double immunofluorescence and confocal LASER scanning fluorescence microscopy (FIG. 4). As positive control LMH cells were infected with USDA-ch.

Infected cells showed a positive signal (Cy5-fluorescence) with the anti-gC MAb and the anti gJ MAb. The specificity of the fluorescence was confirmed since the Cy5-fluorescence was only present in those cells that reacted with the polyclonal chicken anti-ILTV serum (FITC-fluorescence) (FIG. 4A). Cells infected with the gJ deletion mutant ADgJ4.1 did also bind antibodies from ILTV infected chickens as well as the gC mab, but did not bind the gJ mab (FIG. 4B) indicating the presence of a recombinant ILTV unable to express gJ. Next the presence or absence of gJ expression was investigated after infection of LMH cells with the rescue mutant gJR4.3.

Double immunofluorescence using the anti-gC MAb (Cy-5 fluorescence) and the rabbit anti-gJ antiserum (FITC-fluorescence) in infected cells confirmed the reconstitution of gJ expression (FIG. 4C). Furthermore, the absence of gJ in ADgJ4.1 virions was assayed by Western blot (FIG. 5). The anti-gJ MAb reacted with high molecular weight proteins from the USDA-ch virions of approximately 85, 115, and 160 kDa (FIG. 5A). In contrast to a previous report where proteins of approximately 85, 115, and 200 kDa were identified as gJ in virions of the 489 ILTV strain, the 200 kDa form of gJ was initially not detected in purified virions of the USDA-ch strain.

The anti-gJ MAb did not react with any of the ADgJ4.1 virion proteins. In contrast, the anti-gC MAb reacted with a protein of approximately 65 kDa in virion preparations of both, the USDA-ch strain and the gJ deletion mutant ADgJ4.1 (FIG. 5B). The lack of binding of the anti-gJ MAb to virion preparations of ADgJ4.1 shows that the corresponding epitope was absent. Therefore, Western blots of virion preparations of USDA-ch and ADgJ4.1 were also probed with a polyclonal serum, the anti-gJ rabbit hyperimmune serum. No binding to virion proteins of ADgJ4.1 was detected in contrast strong reactions with the three species of gJ of 85, 115, and 160 kDa in the USDA-ch virion preparation were observed (FIG. 5C). Incubation with preimmune serum from the same rabbit serving as specificity control resulted in very faint unspecific reactions. Western blots of virion preparations of USDA-ch and CK cell cultures infected with USDA-ch, ADgJ4.1, BDgJ3.2, and the gJ-rescue virus gJR4.3 were also tested for the presence of gJ with a polyclonal rabbit anti-gJ serum (FIG. 5D). Non-infected CK cells served as a negative control. Adjustment of electrophoresis and transfer conditions to favor detection of high molecular weight proteins was successful in resolving the appearance of the different forms of gJ.

The polyclonal rabbit serum recognized proteins at a molecular weight of approximately 85, 115, 160, and 200 kDa in purified virions of USDA-ch (FIG. 5D, lane 1) and in cell cultures infected with USDA-ch and the rescue mutant gJR4.3 (FIG. 5D, lanes 3 and 4). In non-infected and in infected cell cultures a band at approximately 45 kDa was observed (FIG. 5D, lanes 2-6) indicating a reactivity of the rabbit serum with a cellular protein.

A side-by-side Western blot was preformed incubating the blot with rabbit pre-immune serum, this blot resulted in very faint reactions indicating that the proteins recognized by the gJ polyclonal rabbit serum were indeed ILTV gJ-specific. These results confirmed the absence of gJ expression in the gJ deletion mutants. As a control, all samples were also probed in parallel with the anti-gC MAb (FIG. 5E). In all samples containing either infected cells (FIG. 5E, lanes 3-6) or virions (FIG. 5E, lane 1) a 65 kDa protein was detected with the anti-gC MAb, which was absent in uninfected cells (FIG. 5E, lane 2), proving the presence of comparable amounts of viral proteins in the samples of infected cells.

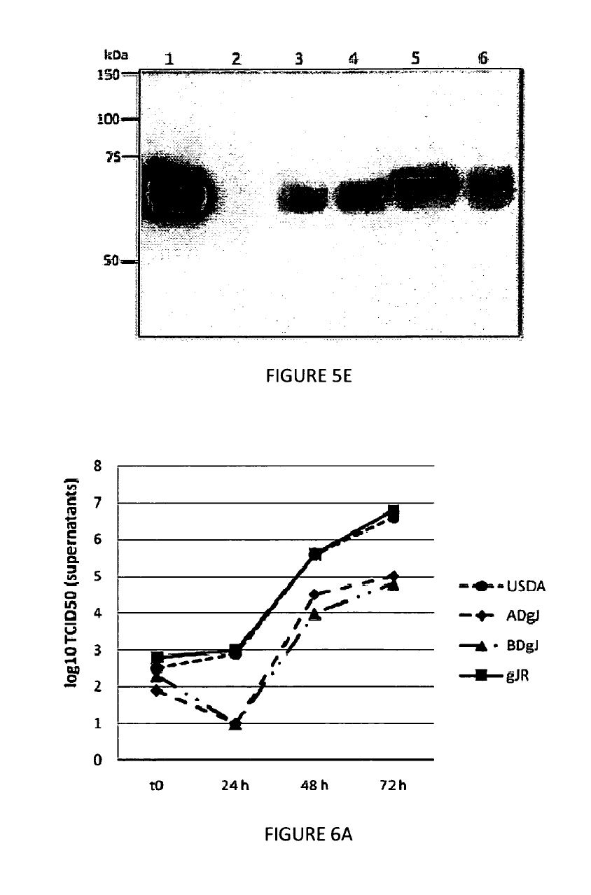

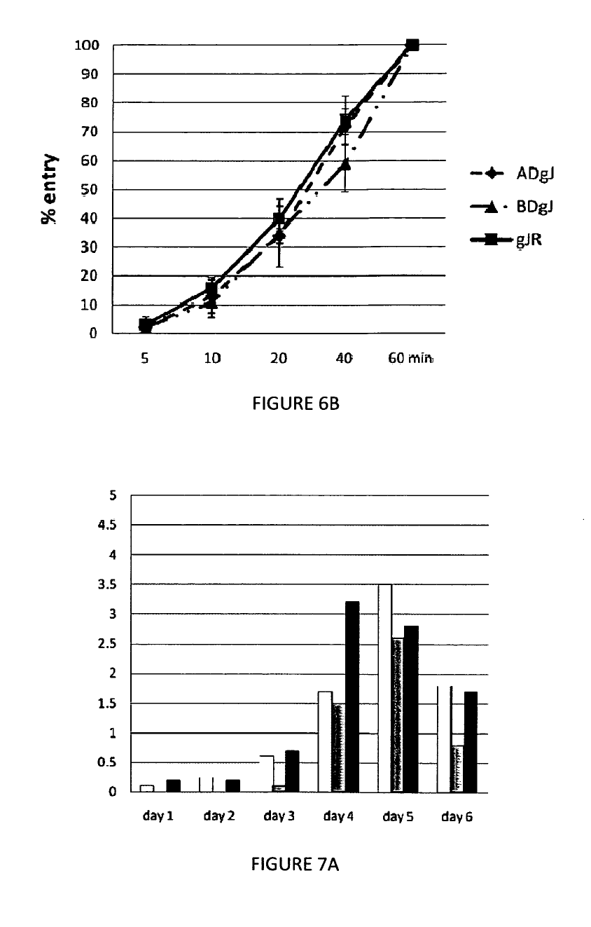

Replication of ILTV gJ deletion mutants in cell culture and chicken embryos. The viral titers in the supernatants of ADgJ4.1 and BDgJ3.2 infected CK cells were reduced by 1.5 to 2 log₁₀ as compared to USDA-ch and the rescue mutant gJR4.3 at 24, 48 and 72 hours p.i. (FIG. 6A). The observed phenotype can be attributed to the absence of gJ expression, since the replication efficiency was fully restored in the virus rescue mutant gJR4.3. (FIG. 6A). Impairment of viral replication by lack of gJ expression can be caused at any step during the process from entry to release. Experiments to investigate whether the virus entry is impaired showed no significant differences in the ability of the USDA-ch, the gJ deletion mutants (ADgJ4.1 and BDgJ3.2) and the rescue mutant (gJR4.3) to enter LMH cells (FIG. 6B). This indicated that viral entry was not detectably affected by the lack of gJ expression.

One of the standard methods to propagate ILTV is in the chorioallantoic membrane (CAM) of chicken embryos (CE). The ability of gJ deletion mutants to replicate in CAM was evaluated and compared to viral titers obtained in CK cells. The viral titers for the rescue mutant gJR4.3 (TCID₅₀ 7.4 log₁₀) and the wildtype USDA-ch (TCID₅₀ 8.0 log₁₀) were 10 to 50 fold higher in CE CAMs (Table 2) than in CK cells (FIG. 6A), respectively. The EGFP expressing gJ-deletion mutant ADgJ4.1 mutant reached a titer of 6.40 log₁₀ after four consecutive passages in CAMs, while the titers of the gJ mutant BDgJ3.2 in CAMs ranged from 2.50 to 1.75 after three consecutive passages (Table 2).

TABLE 2
TCID₅₀ titers of ILTV gJ deletion mutants after
passage on CAM of SPF embryos:

| | P1 | P2 | P3 | P4 |
|---|---|---|---|---|
| USDA (ch) | 8.0^{A} | nd^{B} | nd | nd |
| gJR4.3 | 7.3 | 5.6 | 7.4 | nd |
| ADgJ4.1 | 4.7 | 4.0 | 6.25 | 6.40 |
| BDgJ3.2 | 2.5 | 2.0 | 1.75 | nd |

^{A}Titers expressed as the log10 of TCID50 in chicken kidney cells,
^{B}Not done.

Attenuation of gJ mutants in chickens and their protection efficiency. As expected, the wild type USDA-ch strain and gJR inoculated chickens developed characteristic signs of the disease from days 3 to 6 p.i. after infection of 4-weeks-old SPF chickens. The most prominent signs of disease were severe conjunctivitis and depression. In contrast, few of the ADgJ4.1 inoculated chickens showed a very mild conjunctivitis on days 4 and 5 p.i., and the BDgJ inoculated chickens showed no clinical signs, similar to the control group.

Viral replication was assayed by quantitative PCR (qPCR) of conjunctiva and tracheal swabs collected on day 4 p.i. (Table 3).

TABLE 3
Detection of viral DNA at day 4 after infection.

Genome copies/sample^{A}

| Virus | Eyelid swabs | Tracheal swabs |
|---|---|---|
| Mock infected | <25^{B} | <25 |
| ADgJ4.1 | <25 | <25 |
| BDgJ3.2 | <25 | <25 |
| gJR4.3 | 4000 | 1000 |
| USDA-ch | 3000 | 700 |

^{A}Samples from the swab samples were investigated using a qPCR (Callison et al, (2003) Avian Dis. 50: 50-54.
^{B}Number of DNA copies.

Since the detection limit of the qPCR assay is 25 copies of viral DNA samples from mock-infected and gJ deletion mutant-infected chickens were considered negative for the presence of viral DNA. In contrast, in samples from chickens infected with the wild-type USDA-ch or the rescue mutant gJR4.3 comparable amounts of viral DNA were detected.

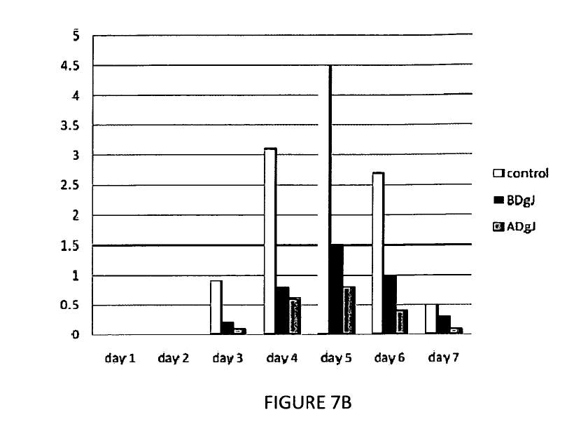

Birds inoculated with the gJ-deletion mutants and the non-infected control birds were challenged with the USDA-ch wildtype virus. Clinical signs of disease were observed in birds infected with the gJ deletion mutants ADgJ4.1 and BDgJ3.2 and the non-infected control birds. The peak of clinical signs was observed between days 4 and 5 after challenge infection. Conjunctivitis and depression were the most prevalent signs of disease. No significant differences were observed in the severity of clinical signs detected in the non-vaccinated/challenged group of chickens and chickens inoculated with ADgJ4.1 and BDgJ3.2 (FIG. 7A).

gJ mutants provide protection after in ovo vaccination. At hatch, no significant differences in hatching rates were observed between the ADgJ4.1 and BDgJ3.2 inoculated groups as compared to the sham inoculated embryos indicating adequate attenuation of gJ deletion mutants for in ovo inoculation. During the rearing period no significant differences to the sham-inoculated group were observed.

Chickens were challenged on day 35 by inoculation of 10⁵ TCID50/chicken conjunctivally and nasally. After challenge, clinical signs were observed from days 1 to 7 and 100% of the non-vaccinated chickens showed severe clinical signs indicating a valid challenge. Of the chickens inoculated with the BDgJ mutant 39% showed clinical signs, while the remaining chickens showed no clinical signs at any time. 14% of the ADgJ4.1 in ovo inoculated chickens showed clinical signs, whereas 86% stayed healthy throughout the experiment (FIG. 7B). These data show that the gJ deletion mutants, in particular ADgJ4.1, when inoculated in ovo, were able to induce protection against ILT after a high-dose challenge infection.

### Example 2

Replacement of green fluorescent protein (GFP) cassette in ILTV-ΔgJgreen with sequence that does not encode ILTV proteins. Design of the recombination DNA for generation of a novel gJ deleted ILTV

From the genome nucleotide sequence of ILTV as stored at Genbank accession number NC₁₃ 006623.1, the coding sequence (CDS) for glycoprotein D (gD) US6 was predicted to be from nucleotide 132675 to 133808, with a 12 nucleotide overlap with the CDS (129739 to 132696) of glycoprotein J, US5. Since the transcription start site has not been mapped for ILTV gD, a longer CDS starting at nucleotide 132504 was considered. In that case, US6 would overlap 192 by with US5. For the design of the novel delta gJ mutant ILTV the promoter sequences upstream of US6 were not modified to assure the expression of US6. To modify the gJ ORF (US5) the 5′ end sequence (2357 bp) of US5 was markedly altered by cutting sections of approximately 10 by and inserting them ten by downstream. This was done 50 times resulting in a nonsense sequence without changing the GC content. In addition, approximately 50 CG and AT exchanges were randomly introduced. The 321 nucleotide fragment from the 3′end of US5, including the earliest possible start codon of US6, plus 130 nucleotides upstream of the potential promoter region were left unchanged. For homologous recombination into the viral genome authentic sequences were added to the 5′ and 3′ ends of the manipulated US5. At the 5′ end a 479 by fragment upstream of the destroyed US5 start codon, comprising 270 nucleotides of the 3′end of US4 and a 209 nucleotide sequence between the stop codon of US4 and the former start codon of US5, was added. At the 3′end a 450 by fragment from the non-overlapping part of US6 was added. The resulting 3876 by DNA fragment was synthesized and cloned in the bacterial plasmid pUC57 (GenScript, Piscataway, N.J.).

Generation of recombinant NΔgJ ILTV

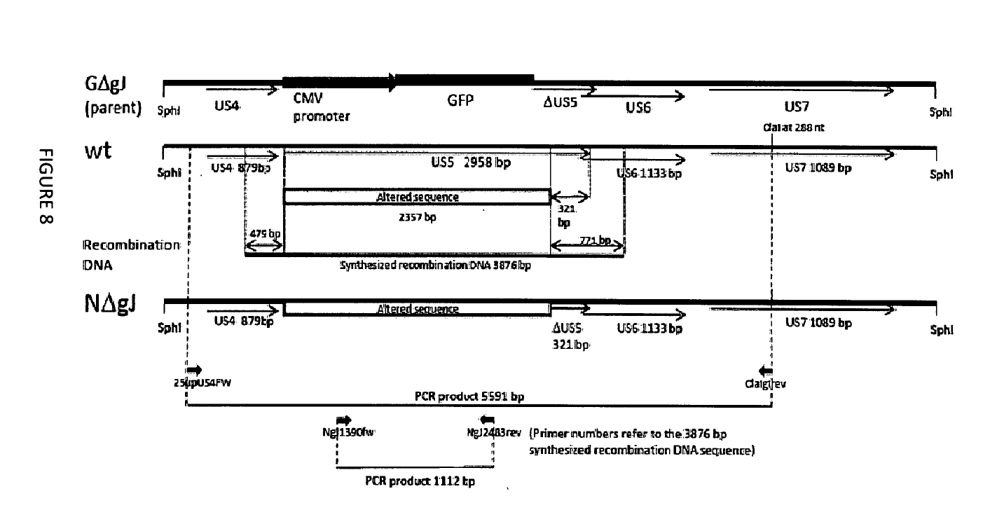

The recombinant plasmid containing the recombinant DNA sequence (3876 bp) was restriction digested with HindIII and EcoRI to release the insert and separated by agarose gel electrophoresis. The 3876 by insert was eluted from the gel and used for cotransfection with viral DNA from the green fluorescent gJ deletion mutant GΔgJ. LMH cells were transfected at 80% confluency using the TransIT mRNA transfection kit (Roche; Indianapolis, Ind.). Five days after transfection, cells were rinsed into the supernatant and stored at −80° C. Serial dilutions were used to infect LMH cells in 6-well plates. Non-fluorescent plaques were identified by live fluorescence microscopy, and non-fluorescent plaques were aspirated under visual control and inoculated into new LMH cell cultures. Plaque purification was repeated once to exclude contamination with parental green fluorescent virus. Plaque purified NΔgJ isolates were inoculated in primary chicken kidney cell cultures and incubated for 3 days at 39° C. Infected cells were rinsed into the supernatant and aliquots were stored at −80° C. One aliquot of each plaque isolate was used to prepare DNA using the QiAmp DNA Blood mini kit. Genotypes of plaque isolates were analyzed by PCR. Using primers 25upUS4FW/ ClaIgIrev (FIG. 8), 5591 by PCR products were obtained from three different plaque isolates of NΔgJ as well as from the USDA control virus DNA, and a 4901 by fragment was obtained using viral DNA from the parent virus GDgJ. The primers bind to regions within the viral genome that lie outside of the recombination region. The binding sites in the mutant NΔgJ must be identical to the original wt virus USDA and are also identical in the parent virus GDgJ, which was originally derived from USDA. Amplification of the 5591 by fragment showed that the primer binding sites are present and that the genomic region between them is of the expected length, which does not differ from USDA, but is different from the parent GDgJ. The PCR products from the NΔgJ isolates were eluted from the agarose gel for cloning and sequencing. In order to confirm presence of the artificial nonsense gJ sequence in the plaque isolates, another PCR was performed using primers NgJ1390fw/NgJ2483rev, which exclusively bind to the nonsense gJ sequence and not the USDA or GΔgJ virus DNA. As expected, 1112 by products (FIG. 8) were amplified only from the NΔgJ plaque isolates and no product was obtained using USDA virus DNA as a template.

Next, the 5591 by PCR products encompassing US4, nonsense US5, and US6 of the NDgJ plaque isolates were cloned and sequenced. The novel delta gJ virus (NΔgJ) virus that does not express the green fluorescent protein and is devoid of any foreign DNA was plaque purified twice, and the genotypes of three individual isolates were confirmed by different PCR using primers that allow differentiation from the parent or wt ILTV. The NΔgJ was propagated in chicken kidney cells. Three plaque-purified viruses have been obtained through 3 passages in CK cells; the plaque-purified viruses have titers ranging from log₁₀ 5.5 to log₁₀ 6.4. This titers are higher than those obtained with the GΔgJ.

The attenuation and protection ability of cell free virus preparations of the NΔgJ strain is tested in broilers and layers. Initially the NΔgJ strain is applied via eye drop at two and six weeks of age. Afterwards, the efficacy of protection when this virus is applied via spray to 1 day old chickens and via in ovo is tested.

Generation and Propagation of FΔGj

Based on the previously generated GFP-expressing gJ deletion mutant (GΔgJ), a recombinant gJ deletion mutant carrying the fusion protein (F) of the Newcastle Disease virus LaSota strain was generated. The GFP-expression cassette was removed and replaced with the fusion gene of NDV LaSota strain. The FΔgJ was rescued after co-transfection with viral DNA from GΔgJ, helper plasmids pRcICP4 and pcUL48 and a recombination DNA fragment. Viruses not expressing GFP were plaque purified twice and the genotypes of three individual isolates were confirmed by PCR using primers that allow differentiation from the parent or wt ILTV. Two of the plaque-purified viruses have been passaged twice in chicken kidney cells reaching titers of log₁₀ 4.6 and log₁₀ 5.12 TCID₅₀ per ml.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

1. A composition comprising an attenuated infectious laryngotracheitis virus (ILTV) comprising a glycoprotein J mutation, wherein the mutation inhibits expression of glycoprotein J.

2. The composition of claim 1, wherein the attenuated ILTV comprises USDA challenge strain USDA-ch.

3. The composition of claim 1, wherein the mutation inhibits the expression of glycoprotein J protein.

4. The composition of claim 3, wherein the mutation comprises a glycoprotein J promoter element mutation and wherein the mutation inhibits a function of the promoter element.

5. The composition of claim 1, wherein the mutation comprises a deletion of a glycoprotein J nucleotide sequence.

6. The composition of claim 5, wherein the mutation comprises a complete or partial deletion of SEQ ID NO:1.

7. The composition of claim 6, wherein the mutation comprises a deletion of nucleotides 1-2291 of SEQ ID NO:1.

8. The composition of claim 6, wherein a reporter protein expression cassette is inserted at the point of the deletion.

9. The composition of claim 8, wherein the reporter protein is green-fluorescent protein.

10. The composition of claim 6, wherein the mutation comprises a deletion of nucleotides 1-2188 of SEQ ID NO:1.

11. The composition of claim 10, wherein a reporter protein expression cassette is inserted at the point of the deletion.

12. The composition of claim 11, wherein the reporter protein is green-fluorescent protein.

13. The composition of claim 10, wherein a viral protein expression cassette is inserted at the point of deletion.

14. The composition of claim 13, wherein the viral protein expression cassette is fusion protein (F) of the Newcastle Disease Virus.

15. The composition of claim 6, wherein the mutation comprises a deletion of at least nucleotides 1-145 of SEQ ID NO:1.

16. The composition of claim 6, wherein the deletion includes nucleotides 2185-2190 of SEQ ID NO:1.

17. The composition of claim 1, wherein the mutation comprises a substitution of the glycoprotein J nucleotide sequence.

18. The composition of claim 17, wherein the substitution comprises a rearranged ILTV sequence.

19. A method of preventing infectious laryngotracheitis virus (ILTV) infection in a subject or population, the method comprising administering to one or more subject an attenuated infectious laryngotracheitis virus (ILTV), wherein the attenuated ILTV is administered in ovo.

20. The method of claim 19, wherein the attenuated ILTV comprises USDA challenge strain USDA-ch.

21. The method of claim 19, wherein the attenuated ILTV comprises a glycoprotein J mutation.

22. The method of claim 21, wherein the mutation inhibits expression of glycoprotein J protein.

23. The method of claim 22, wherein the mutation comprises a glycoprotein J promoter element mutation, wherein the mutation inhibits a function of the promoter element.

24. The method of claim 21, wherein the mutation comprises a deletion of a glycoprotein J nucleotide sequence.

25. The method of claim 24, wherein the mutation comprises a complete or partial deletion of SEQ ID NO:1.

26. The method of claim 25, wherein the mutation comprises a deletion of nucleotides 1-2291 of SEQ ID NO:1.

27. The method of claim 24, wherein a reporter protein expression cassette is inserted at the point of the deletion.

28. The method of claim 27, wherein the reporter protein is green-fluorescent protein.

29. The method of claim 24, wherein a viral protein expression cassette is inserted at the point of deletion.

30. The method of claim 29, wherein the viral protein expression cassette is fusion protein (F) of the Newcastle Disease Virus.

31. The method of claim 25, wherein the mutation comprises a deletion of nucleotides 1-2188 of SEQ ID NO:1.

32. The method of claim 24, wherein a reporter protein expression cassette is inserted at the point of the deletion.

33. The method of claim 32, wherein the reporter protein is green-fluorescent protein.

34. The method of claim 25, wherein the mutation comprises a deletion of at least nucleotides 1-145 of SEQ ID NO:1.

35. The method of claim 25, wherein the deletion comprises nucleotides 2185-2190 of SEQ ID NO:1.

36. The method of claim 21, wherein the mutation comprises a substitution of the glycoprotein J nucleotide sequence.

37. The method of claim 36, wherein the substitution comprises a rearranged ILTV sequence.

38-39. (canceled)

40. The composition of claim 1, formulated as an in ovo vaccine.

41. The method of claim 19, wherein the population comprises avian subjects, the method comprising administering in ovo an attenuated ILTV to one or more eggs that give rise to one or more subjects of the population.

42-46. (canceled)


SEQUENCE LISTING (24 sequences)

SEQ ID NO: 1 - DNA, 2958 bp, Artificial Sequence
  Synthetic Construct
atggggacaatgttagtgttgcgccttttcctacttgcagtagcggacgcggcgttgccgaccggcagattctgccgagtttggaaggtgcctccgggaggaaccatccaagagaacctggcggtgctcgcggaatcgccggtcacgggacacgcgacatatccgccgcctgaaggcgccgtcagctttcagatttttgcggacacccctactttgcgcattcgctacggcgctacggaggacgaacttgcactggagcgcgggacgtccgcctcagacgcggacaacgtgacattttcgctgtcatatcgcccgcgcccagaaattcacggagcatacttcaccataggggtattcgctactggccagagcacggaaagcagctattcggtcatcagtcgggtcttagttaacgcctctctggaacggtccgtgcgcctggaaacgccgtgcgatgaaaattttttgcagaacgagcctacatggggctcgaagcgttggttaggccccccgtcgccttatgtgcgagataacgatgtcgccgtgttgacaaaagcgcagtacattggggagtgctactccaactcggcggcccagacggggctcacgtctctcaacatgacctttttctattcgcctaaaagaatagtaaacgtcacgtggacaaccggcggcccctccccctcgcgcataacggtatactcgtcgcgggagaacgggcagcccgtgttgaggaacgtttctgacgggttcttggttaagtacactcccgacattgacggccgggccatgataaacgttattgccaattattcgccggcggactccggcagcgtcctcgcgtttacggcctttagggaaggaaaactcccatccgcgattcaactgcaccggatagatatgtccgggactgagccgccggggactgaaacgaccttcgactgtcaaaaaatgatagaaaccccgtaccgagcgctcgggagcaatgttcccagggacgactctatccgtccgggggccactctgcctccgttcgataccgcagcacctgatttcgatacaggtacttccccgacccccactaccgtgccagagccagccattactacactcataccgcgcagcactagcgatatgggattcttctccacggcacgtgctaccggatcagaaactctttcggtacccgtccaggaaacggatagaactctttcgacaactcctcttacccttccactgactcccggtgagtcagaaaatacactgtttcctacgaccgcgccggggatttctaccgagaccccgagcgcggcacatgaaactacacagacccagagtgcagaaacggtggtctttactcagagtccgagtaccgagtcggaaaccgcgcggtcccagagtcaggaaccgtggtattttactcagactccgagtactgaacaggcggctcttactcagacgcagatcgcagaaacggaggcgttgtttactcagactccgagtgctgaacagatgacttttactcagactccgggtgcagaaaccgaggcacctgcccagaccccgagcacgatacccgagatatttactcagtctcgtagcacgccccccgaaaccgctcgcgctccgagcgcggcgccggaggtttttacacagagttcgagtacggtaacggaggtgtttactcagaccccgagcacggtaccgaaaactactctgagttcgagtactgaaccggcgatttttactcggactcagagcgcgggaactgaggcctttactcagacttcgagtgccgagccggacactatgcgaactcagagtactgaaacacactttttcactcaggccccgagtacggtaccgaaagctactcagactccgagtacagagccggaggtgttgactcagagtccgagtaccgaacctgtgcctttcacccggactctgggcgcagagccggaaattactcagaccccgagcgcggcaccggaggtttatactcggagttcgagtacgatgccagaaactgcacagagcacacccctggcctcgcaaaaccctaccagttcgggaaccgggacgcataatactgaaccgaggacttatccagtgcaaacgacaccacatacccagaaactctacacagaaaataagactttatcgtttcctactgttgtttcagaattccatgagatgtcgacggcagagtcgcagacgcccctattggacgtcaaaattgtagaggtgaagttttcaaacgatggcgaagtaacggcgacttgcgtttccaccgtcaaatctccctatagggtagaaactaattggaaagtagacctcgtagatgtaatggatgaaatttctgggaacagtcccgccggggtttttaacagtaatgagaaatggcagaaacagctgtactacagagtaaccgatggaagaacatcggtccagctaatgtgcctgtcgtgcacgagccattctccggaaccttactgtcttttcgacacgtctcttatagcgagggaaaaagatatcgcgccagagttatactttacctctgatccgcaaacggcatactgcacaataactctgccgtccggcgttgttccgagattcgaatggagccttaataatgtttcactgccggaatatttgacggccacgaccgttgtttcgcataccgctggccaaagtacagtgtggaagagcagcgcgagagcaggcgaggcgtggatttctggccggggaggcaatatatacgaatgcaccgtcctcatctcagacggcactcgcgttactacgcgaaaggagaggtgcttaacaaacacatggattgcggtggaaaacggtgctgctcaggcgcagctgtattcactcttttctggacttgtgtcaggattatgcgggagcatatctgctttgtacgcaacgctatggaccgccatttatttttga

SEQ ID NO: 2 - DNA, 31 bp, Artificial Sequence
  Synthetic Construct
agcggatccatggggacaatgttagtgttgc

SEQ ID NO: 3 - DNA, 63 bp, Artificial Sequence
  Synthetic Construct
gtgcggccgcctaatggtgatggtgatggtgacttcctctaaaataaatggcggtccatagcg

SEQ ID NO: 4 - DNA, 26 bp, Artificial Sequence
  Synthetic Construct
gcagaattcagttgcgctgagtaccg

SEQ ID NO: 5 - DNA, 27 bp, Artificial Sequence
  Synthetic Construct
cgtcccgggcgaaatacgctgcacgcc

SEQ ID NO: 6 - DNA, 30 bp, Artificial Sequence
  Synthetic Construct
cgagcatgcatctccctatagggtagaaac

SEQ ID NO: 7 - DNA, 27 bp, Artificial Sequence
  Synthetic Construct
cctaagcttatgagcgtgaggcgtggc

SEQ ID NO: 8 - DNA, 27 bp, Artificial Sequence
  Synthetic Construct
gcacccgggccagatatacgcgttgac

SEQ ID NO: 9 - DNA, 22 bp, Artificial Sequence
  Synthetic Construct
cgtcatagagcccaccgcatcc

SEQ ID NO: 10 - DNA, 26 bp, Artificial Sequence
  Synthetic Construct
gctacccgggcttcagttgcgctgag

SEQ ID NO: 11 - DNA, 31 bp, Artificial Sequence
  Synthetic Construct
cgatcccgggcgtggcatgtaggaagaaacc

SEQ ID NO: 12 - DNA, 30 bp, Artificial Sequence
  Synthetic Construct
gctgaattcctcggcgaaatacgctgcacg

SEQ ID NO: 13 - DNA, 24 bp, Artificial Sequence
  Synthetic Construct
gcagaattccatgagatgtcgacg

SEQ ID NO: 14 - DNA, 27 bp, Artificial Sequence
  Synthetic Construct
cacggatccatggaagaagaatcttcc

SEQ ID NO: 15 - DNA, 33 bp, Artificial Sequence
  Synthetic Construct
gctgcggccgcttagggcataggtgtatcaagg

SEQ ID NO: 16 - DNA, 20 bp, Artificial Sequence
  Synthetic Construct
gtcttcactcgatatcatgg

SEQ ID NO: 17 - DNA, 20 bp, Artificial Sequence
  Synthetic Construct
gtcattattgacgtcaatgg

SEQ ID NO: 18 - DNA, 20 bp, Artificial Sequence
  Synthetic Construct
cgtgctgctgcccgacaacc

SEQ ID NO: 19 - DNA, 21 bp, Artificial Sequence
  Synthetic Construct
cagaagacgatcgatgagtgc

SEQ ID NO: 20 - DNA, 19 bp, Artificial Sequence
  Synthetic Construct
ggcaatggatccctggtgc

SEQ ID NO: 21 - DNA, 21 bp, Artificial Sequence
  Synthetic Construct
ctgttcccagaaatttcatcc

SEQ ID NO: 22 - DNA, 21 bp, Artificial Sequence
  Synthetic Construct
cgaacctgtgcctttcacccg

SEQ ID NO: 23 - DNA, 23 bp, Artificial Sequence
  Synthetic Construct
cgccagggttttcccagtcacga

SEQ ID NO: 24 - DNA, 24 bp, Artificial Sequence
  Synthetic Construct
cacacaggaaacagctatgaccat